(19) United States
(12) Patent Application Publication
(10) Pub. No.: US 2008/0286355 A1
(43) Pub. Date: Nov. 20, 2008

(54) Protease Inhibitors For the Treatment of Digestive Pathologies

(76) Inventor: Lionel Bueno, Aussonne (FR)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/586,085

(22) PCT Filed: Jan. 18, 2005
(86) PCT No.: PCT/FR2005/000108

§ 371 Date: Aug. 7, 2008

(30) Foreign Application Priority Data
Jan. 19, 2004 (FR) 0400446

(51) Int. Cl. A61K 9/48; A61K 38/57; A61P 1/00; A61K 36/48

(52) U.S. Cl. 424/456; 424/757

(57) ABSTRACT

The invention relates to compositions and methods for the treatment of intestinal pathologies. The invention also relates to compositions and methods which can be used to regulate the paracellular permeability of the intestinal epithelium. The inventive compositions and methods are based in particular on the use of protease inhibitors which modulate the opening of the tight junctions of the intestinal epithelium. The invention can be used for preventive or curative treatment of different pathologies, such as functional digestive disorders (FGID), in particular intestinal functional disorders (IFD) causing hyperalgesia and in particular irritable bowel syndrome (IBS), in mammals, particularly humans.

The invention relates to compositions and methods for the treatment of intestinal pathologies. The invention also relates to compositions and methods which can be used to regulate the paracellular permeability of the intestinal epithelium. The compositions and methods of the invention are based in particular on the use of protease inhibitors which modulate the opening of the tight junctions of the intestinal epithelium. The invention can be used for preventive or curative treatment of varied pathologies, such as functional digestive (gastroinstestinal) disorders (FGID), more particularly intestinal functional disorders (IFD), and in particular irritable bowel syndrome (IBS), hyperalgesia and other abdominal pain, etc., in mammals, particularly humans.

The intestinal epithelium is the site of very important exchanges between the exterior medium and the body. Said exchanges can take place either across the cells of the epithelium, or through parallel networks. For instance, water and electrolyte transport, or else absorption of small molecules (molecular weight generally less than about 1000 Da) by the gastric, intestinal or colon mucosa, takes place by a transcellular route, across epithelial cells or enterocytes. On the other hand, the absorption of large molecules and the passage of antigens, toxins or immune cells occurs mainly by the paracellular route, at the level of “tight junctions” which are located between the epithelial cells.

Epithelial tight junctions (TJ) are structures which link the cells lining mucosal epithelia (gastrointestinal tract, lungs). Said structures ensure and control paracellular transport across the epithelium, from the exterior to the submucosa, of various macromolecules (allergens, irritants, toxins, microorganisms). Said structures also enable the migration of immune cells (e.g., immunocytes) towards the exterior (digestive tube). Tight junctions are flexible structures composed of a complex assembly of transmembrane proteins (occluding, claudins) and cytoplasmic proteins. (zona occludens proteins ZO-1, ZO-2, ZO-3, proteins AF7, cingulin or 7H6, etc.), which are associated with components of the cytoskeleton (actin, myosin filaments, etc.).

Among the functional digestive (gastroinstestinal) disorders (FGID), abdominal pain without defecation problems can be distinguished from intestinal functional disorders. Abdominal pain with normal defecation can be caused by allergy or food intolerance or can occur in celiac disease for example. Intestinal functional disorders affect 15 to 20% of the population and are characterized by dyspeptic and/or intestinal symptoms for which no organic cause has yet been identified and which require a specific treatment. The common feature of functional digestive disorders which meet the ROME criteria is postprandial abdominal pain, whether it be localized in the upper (dyspepsia) or lower abdomen. A specific disorder affecting the lower abdomen is irritable bowel syndrome (IBS). The pain associated with intestinal functional disorders is usually relieved by defecation. The pathophysiology of functional digestive disorders is poorly understood although recent clinical investigations have revealed that affected patients have a lower pain perception threshold to distention (visceral hyperalgesia) resulting from a state of digestive hypersensitivity. This appears to arise firstly from a sensitization of parietal mechanoreceptors by proinflammatory mediators. In fact, the existence of modifications in the structure or density of certain immune cells in the submucosa, particularly the colon, argues in favor of an altered immune equilibrium with the resident microflora. Ileum and colon biopsies of subjects with IBS show an increased density of mast cells and enterochromaffin cells. This digestive microinflammatory state promotes sensitization of primary afferent nerve endings which in turn might facilitate transmission of nociceptive messages to the brain. Indeed, functional imaging studies reveal characteristic alterations of the cortical projection areas of these nerve signals in subjects with FGID.

Said process of intestinal sensitization to pain can be induced by factors such as stress, pathogens, allergens, enzymes like trypsin or tryptase for example, bile salts, xenobiotics, chemical molecules of the type glycerol, TNBS or taurocholate for example and/or sequelae of infection or surgery. However, the mechanisms whereby said factors trigger the sensitization phenomenon have not been clearly documented in vivo. When allergens, pathogens and/or chemical molecules for example are absorbed, they enter into contact with the intestinal epithelial wall which prevents them from entering the body and coming into contact with immune cells. However, in order for sensitization to develop, some allergens, pathogens and/or chemical molecules must be able to cross this epithelium to interact with immune cells. The conditions in which this passage is possible in vivo remain obscure. Thus, while some studies on in vitro cell cultures suggest a role of tight junctions in this process, there is no evidence for a role of said junctions in vivo in the development of sensitization. Likewise, while Coremans et al. (Ital J Gastroenterol 1991 (8) S 1: 39-40) suggest a link between intracolonic infusion of bile salts and abdominal pain in a subject with irritable bowel syndrome, no results are presented demonstrating a correlation between tight junctions and sensitization of the intestinal epithelium to pain.

While malabsorption of bile salts is observed in only a small proportion of subjects with irritable bowel syndrome, alterations of transepithelial permeability have been described in subjects who developed IBS secondary to gastroenteritis (Spiller et al. Gut 2000; 47: 804-11) but this study makes no mention of modifications in paracellular permeability which was not investigated. Experimental studies have found a correlation between irritable bowel syndrome and an increased presence of microorganisms in the submucosa (AGA. 1999), just as a relationship has been observed between chronic intestinal inflammatory disorders or intestinal functional disorders and changes in the degree of immune activation (inflammation) of the intestinal wall. However, these preliminary findings have not been confirmed or have not given rise to novel therapeutic approaches.

The invention results from the demonstration that bacterial proteases released in the colon lumen activate PAR receptors (proteinase-activated receptors), located on the epithelial cell membrane, which modulate the opening of tight junctions, said opening leading to a state of hyperalgesia.

The bacterial origin of the aforementioned proteases was demonstrated by the fact that a 10-day course of treatment with an oral antibiotic cocktail destroying the flora reduced the permeability of tight junctions, said permeability being restored by intracolonic infusion of a supernatant of normal colon contents. The inventors also show that a cocktail of protease inhibitors infused in the colon lumen decreased colonic paracellular permeability and visceral sensitivity to distention. The in vivo role of intestinal epithelial tight junctions in the process of pain sensitization has also been demonstrated in patent application WO 03/077893. The “opening” of tight junctions of the colon epithelium by different molecules or by stress results in spontaneous hyperalgesia or hypersensitivity to distention (characteristic feature of IBS). The results obtained in the scope of this invention have established for the first time and in a surprising manner that the opening of tight junctions induces a state of sustained, delayed hyperalgesia.

The invention results from the development of novel therapeutic strategies for treating intestinal pathologies, based on modulating the paracellular permeability of the intestinal epithelium with the aid of protease inhibitors. In particular, the invention provides a therapeutic approach to intestinal pathologies based on the use of protease inhibitors allowing to control the opening of tight junctions of the intestinal epithelium. For instance, said protease inhibitors make it possible to modulate cytoskeletal tension of intestinal epithelial cells or to directly regulate, preferably reduce, even block, the opening of intestinal epithelial tight junctions. Said approach therefore enables control of the opening and closing of intestinal epithelium tight junctions, without necessarily requiring de novo protein synthesis and/or major protein and/or structural degradations in the epithelium. Said strategy makes it possible to regulate intestinal epithelial permeability in a specific, fine and reactive way, and hence to act on the passage of allergens, pathogens and/or chemical molecules towards immune cells. Said strategy is particularly adapted to obtaining a rapid biological effect which can be controlled over time (reversible).

In this regard, the results presented hereinbelow show that a substance which can relax epithelial tight junctions (PAR-2 receptor activator peptides such as the peptide SLIGRL) trigger delayed hyperalgesia, hypersensitivity to distention and an increase in colon permeability. The results presented in the examples also show that blocking this increase in paracellular permeability with a protease inhibitor or a mixture (cocktail) of protease inhibitors inhibits or reduces this hyperalgesia characteristic of FGID and in particular of IBS.

Therefore, a first object of the invention is more particularly based on the use of at least one protease inhibitor, for preparing a medicament for the preventive or curative treatment of functional digestive disorders. The invention also relates to said use for preparing a medicament for the preventive or curative treatment of hyperalgesia occurring in intestinal pathologies. The protease inhibitor is preferably an inhibitor of intracolonic proteases.

Another object of the invention is based on a method for the preventive or curative treatment of intestinal pathologies characterized by a state of hyperalgesia, comprising administering to a subject an effective amount of at least one protease inhibitor.

Protease inhibitors appear to act by controlling the opening of tight junctions of the intestinal epithelium. In particular, said inhibitors are inhibitors which modulate cytoskeletal tension in intestinal epithelial cells or inhibitors which decrease, or block, the opening of intestinal epithelial tight junctions.

The invention is thus based on the use of protease inhibitors modulating the tension and the state of contraction of the cytoskeleton of intestinal epithelial cells or preventing excessive opening of tight junctions which leads to hyperalgesia or hypersensitivity to intestinal distention.

The proteins composing tight junctions are associated with the cytoskeleton of the cells they link together. The invention proposes that the tension of the cytoskeleton or the opening of tight junctions can be modulated in subjects with intestinal diseases or disorders so as to act in a non-destructive and transient manner on the permeability of their intestinal epithelium. Thus, cytoskeletal contraction should promote the opening of tight junctions, whereas cytoskeletal relaxation (or inhibition of contraction) should promote the closing of tight junctions. It is also possible to directly modulate tight junctions, in particular the proteins composing same, by reducing or blocking the opening thereof.

Preferably, then, the invention makes use of protease inhibitors which modulate the contraction of the cytoskeleton of intestinal epithelial cells (particularly human) or which control the opening of tight junctions of the intestinal epithelium (particularly human). Depending on the condition to be treated, one uses protease inhibitors which inhibit the contraction of the cytoskeleton of intestinal epithelial cells, or which activate or promote same.

A protease inhibitor is considered to modulate cytoskeletal tension when it modulates the opening of tight junctions. An inhibitory effect on the contraction or tension of actin and/or myosin filaments does not necessarily have to be complete or total, but it suffices that it reduces cytoskeletal contraction or tension enough to reduce the opening of tight junctions.

Reduction of the opening of tight junctions preferably corresponds to a minimum decrease of approximately 25%, advantageously approximately 30%, even more preferably to a decrease of approximately 50% in the paracellular permeability of the intestinal epithelium. Paracellular permeability can be measured with the aid of a tracer such as ⁵¹Cr-EDTA which, after entering the circulation, is assayed in the 24-hour urine (see Example 1).

The protease inhibitors which are used are preferably inhibitors acting on serine proteases and/or metalloproteases. Said inhibitors are particular active for reducing the action of bacterial proteases on colon permeability.

Advantageously, the protease inhibitors used are molecules, which can be in isolated form or in the form of a cocktail, combination, biological extracts, and the like. Said molecules can be synthetic, semi-synthetic or biological, particularly of animal, viral, plant or bacterial origin.

Examples of protease inhibitors include in particular selective or nonselective inhibitors of serine proteases [serpine and derivatives thereof, aprotinin, N-tosyl-L-phenylalanylchloromethyl ketone (TPCK), dichloroisocoumarin, nexin-1, AEBSF-HCl, Antipain, benzamidine, leupeptin, TLCK, ovomucoid, phenylmethyl sulfonyl fluoride (PMSF), PEFABLOC® and soy bean extracts] and metalloproteases (amastatin, arphamenin, bestatin, diprotin A, phosphoramidon) as well as nonspecific molecules used as antivirals (amprenavir, indinavir, lopinavir, ritonavir, saquinavir, nelfinavir and atazanavir).

The protease inhibitors can be used alone or in combination and/or in association with other active agents, such as for example other active substances used in the treatment of irritable bowel syndrome.

Thus, the protease inhibitors are optionally used in combination and/or in association with compounds which decrease or block the opening of tight junctions of the intestinal epithelium, in particular by modulating cytoskeletal tension, or which increase the opening thereof.

The activity of said compounds can be direct or indirect, that is to say, directed to the cytoskeletal components themselves or to the regulators of cytoskeletal tension. While not by way of limitation, preferred compounds are compounds which act directly on cytoskeletal tension. In addition, one prefers compounds which display selective activity on cytoskeletal tension, that is to say, typically compounds which do not directly affect the structure of the proteins composing the tight junctions. Likewise, the protease inhibitor according to the invention preferably does not directly affect the structure of the proteins composing the tight junctions.

Different types of compounds can be used in combination with the protease inhibitors in the scope of the invention. Thus, in the spirit of the invention, the term “compound” must be understood in the broad sense, that is to say, as designating any agent, substance, composition, condition, treatment or method enabling a modulation of the opening of intestinal epithelial tight junctions. Advantageously it is an agent (e.g., a molecule) or a combination or association of agents. Examples of such compounds may be found in international patent application WO 03/077893. In particular they are inhibitors of myosin light chain kinase (MLCK). A particular example of selective MLCK inhibitor is the compound ML-7 {1-(5-iodonaphthalene-1-sulfonyl)-1H-hexahydro-1,4-diazepine} (Makishima M. et al., FEBS Lett. 1991; 287: 175). Other particular examples of such inhibitors are the compound ML-9 (Wilson D P. et al., J Biol. Chem. 2001; 13: 165) or other nonselective compounds: wortmannin (Warashina A., Life Sci 2000; 13: 2587-93), H-7 (Piao Zf et al., Mol Cell Biol Res Commun 2001; 4: 307-12) and KT 7692 (Warashina A., Life Sci 2000; 13: 2587-93). Other targets of compounds acting on cytoskeletal tension which can be used in combination with the protease inhibitors are in particular the myosin binding proteins, such as for example cingulin, or the junction molecules, such as cadherin-E, catenin-α or desmosomes. Modulation of the activity or expression of said proteins enables a regulation of cytoskeletal tension, in the scope of the invention. It is also possible to use protease inhibitors combined with compounds which inhibit the synthesis of proteins or other molecules ensuring the link between cytoskeletal proteins and tight junction proteins. In the scope of the invention it is also possible to use inhibitors of mitogen-activated kinases (MAPKK), in particular kinase MEK1 or kinase-PI3, such as compounds PD098,059 {2-(amino-3-methoxyphenyl)-4H-1-benzopyran-4-one} (Alessi et al., J. Biol. Chem. 1995; 270: 27589) or LY294002 {2-(4-morpholinyl)-8-phenyl-1(4H)-benzopyran-4-one} (Vlahos et al., J. Biol. Chem 1994; 269: 5241). Other molecules which can be used to indirectly regulate cytoskeletal tension are the growth factors, such as hepatic growth factor (HGF), endothelial growth factor (EGF) or certain cytokines that can be released by immune cells, such as interleukins-1, -4, -13, or factors like IGF-1 or gamma interferon. Another approach by which to indirectly regulate cytoskeletal tension is based on the use of the peptide GLP2 (glucagon-like peptide 2) or derivatives thereof, which can modify intestinal epithelial permeability by an indirect effect on cytoskeletal contraction. Similarly, some molecules acting on receptors located at the apical pole of epithelial cells (e.g., proteinase receptors; PAR-2) can act indirectly on the cytoskeleton. Other examples of active agents are the anticholinergic compounds, prokinetics, antidiarrheals, modifiers of intestinal motility, and the like. These different agents can be used in therapeutic combination, and administered separately, in combination, spread out over time or concomitantly.

Another object of the invention is based on a product, a cocktail or a pharmaceutical association comprising at least one protease inhibitor and at least one other active agent selected in the group consisting of anticholinergic compounds, prokinetic substances, antidiarrheals, laxatives or modifiers of motility, visceral sensitivity (or digestive sensitivity), in view of a use which is combined, separate or spread out over time.

The invention can be used for the treatment or management of pathologies or disorders of the digestive system characterized by a state of hyperalgesia, particularly functional intestinal disorders, chronic intestinal inflammatory diseases (CIID), food intolerance (allergies, formulations, etc.) characterized by chronic abdominal pain. The invention is particularly adapted to the preventive or curative treatment of hyperalgesia and in particular of irritable bowel syndrome (IBS) regardless of its type (constipation, diarrhea or a combination of the two), but also chronic abdominal pain that is not classified as IBS, such as functional abdominal pain without defecation disorder (FAP: Functional Abdominal Pain) and pain related to food intolerance and celiac disease. The invention can be used preventively in subjects with a predisposition or susceptibility to this type of disorder, or in a curative manner, for example during acute episodes or over longer periods. The compositions and methods of the invention make it possible to alleviate the subjects' suffering and attenuate the symptoms or the cause of said disorders.

As a matter of fact, the invention demonstrates in a surprising manner that suppressing the increase in paracellular permeability associated with the activation of PAR receptors (for example the PAR-2 receptor) prevents the development of visceral hyperalgesia.

A particular object of the invention is based on the use of a protease inhibitor such as defined hereinabove for preparing a medicament for controlling, in particular for reducing, the paracellular permeability of the intestinal epithelium in subjects with intestinal diseases characterized by a state of hyperalgesia, particularly chronic inflammatory diseases characterized by a submucosal accumulation of immune cells (for example mast cells and/or enterochromaffin cells), by a heightened sensitivity of parietal mechanoreceptors and possibly by infiltration of colonic bacteria into the submucosal layer, for example hyperalgesia and in particular irritable bowel syndrome.

Another particular object of the invention is based on the use of a protease inhibitor such as defined hereinabove for preparing a medicament for reducing sensitization to allergens, pathogens and/or chemical molecules in subjects suffering from or vulnerable to functional intestinal disorders, in particular intestinal pathologies characterized by a submucosal accumulation of immune cells, particularly mast cells and/or enterochromaffin cells for example, by a heightened sensitivity of parietal mechanoreceptors and possibly by infiltration of colonic bacteria into the submucosal layer, for example hyperalgesia and in particular irritable bowel syndrome.

Another particular object of the invention is based on the use of a protease inhibitor such as defined hereinabove for preparing a medicament for reducing transepithelial migration of immune cells and accumulation thereof in the submucosal layer in subjects with an intestinal functional pathology, particularly an intestinal pathology inducing visceral hyperalgesia, for example irritable bowel syndrome, characterized by a submucosal accumulation of immune cells, particularly mast cells and/or enterochromaffin cells, by a heightened sensitivity of parietal mechanoreceptors and possibly by infiltration of colonic bacteria into the submucosal layer.

The invention also relates to methods for treating the aforementioned conditions, comprising administering to a subject with an intestinal pathology or susceptible to intestinal pathologies, a protease inhibitor or treatment such as defined hereinabove. Preferably, the protease inhibitor or the treatment is administered in a dose which is effective to reduce paracellular permeability of the intestinal epithelium and/or to reduce sensitivity to pain and/or to reduce transepithelial migration of allergens, toxins, irritants or microorganisms and hence the accumulation of immune cells in the submucosal layer of the intestine.

The protease inhibitor can be administered by different routes and in different forms. For instance, the protease inhibitor can be a liquid or solid formulation, typically in the form of a tablet, gelatin capsule, capsule, ampoule or oral solution, a solution for injection, and the like. Compounds formulated for oral administration (oral solutions, tablets, ampoules, gelatin capsules, capsules, syrups, etc.) or rectal administration (suppository) are preferred. Capsule or gelatin capsule formulations which release their contents by microbial digestion in the colon are particularly preferred, when this is possible. Of course, other formulations are possible, such as injections (intraperitoneal, intradermal, subcutaneous, intramuscular, intravenous, intra-arterial, etc.), ointments, gels, and the like.

Another object of the invention is a pharmaceutical composition comprising at least one protease inhibitor and a pharmaceutically acceptable excipient, said composition preferably being formulated for oral or rectal administration. Preferably, the composition is formulated as a suppository, or as a capsule or gelatin capsule which releases its contents by microbial digestion in the colon.

Other aspects and advantages of the invention will become apparent in the following examples, which are given for purposes of illustration and not by way of limitation.

### LEGENDS TO THE FIGURES

FIG. 1: Effect of increasing doses of a PAR-2 receptor activator peptide (SLIGRL) on absorption of a macromolecule (⁵¹Cr-EDTA) expressed as percentage recovery in 24-hour urine of rats.

FIG. 2: Paracellular permeability in mouse colon. Effects of different bacterial protease inhibitors (mean ±SD; n=12):

Protease inhibitor cocktail (½ packet)
Cysteine protease inhibitor (100 μg/ml)
Serine protease inhibitor (100 and 500 μg/ml)
Matrix metalloprotease inhibitors (100 μg/ml)

FIG. 3: Changes in colon paracellular permeability induced by intraluminal infusion of trypsin and of supernatant of colon contents in normal mice (controls) and after a 10-day treatment with an oral antibiotic cocktail (neomycin 2 mg/kg/day+ampicillin 1 mg/kg/day) in mice (mean ±SD; n=12).

FIG. 4: Reduction of rectal sensitivity to distention induced by intracolonic infusion of a protease inhibitor cocktail in rats (mean ±SD; n=8).

FIG. 5: Effect of intracolonic infusion of trypsin on rectal sensitivity to distention in rats (mean ±SD; n=8).

### EXAMPLES

### Example 1

### Reduction of Rectal Hyperalgesia by a Blocker of Tight Junctions

The intestinal epithelium contains structures linking epithelial cells which ensure the controlled passage of immune cells into the intestinal submucosa. This example shows that some molecules known to increase intestinal paracellular permeability such as SLIGRL promote the accumulation of immune cells (mast cells, enterochromaffin cells) in the intestinal submucosa and that this effect can be prevented (e.g., inhibited, reduced) by intracolonic treatment with a blocker of tight junctions.

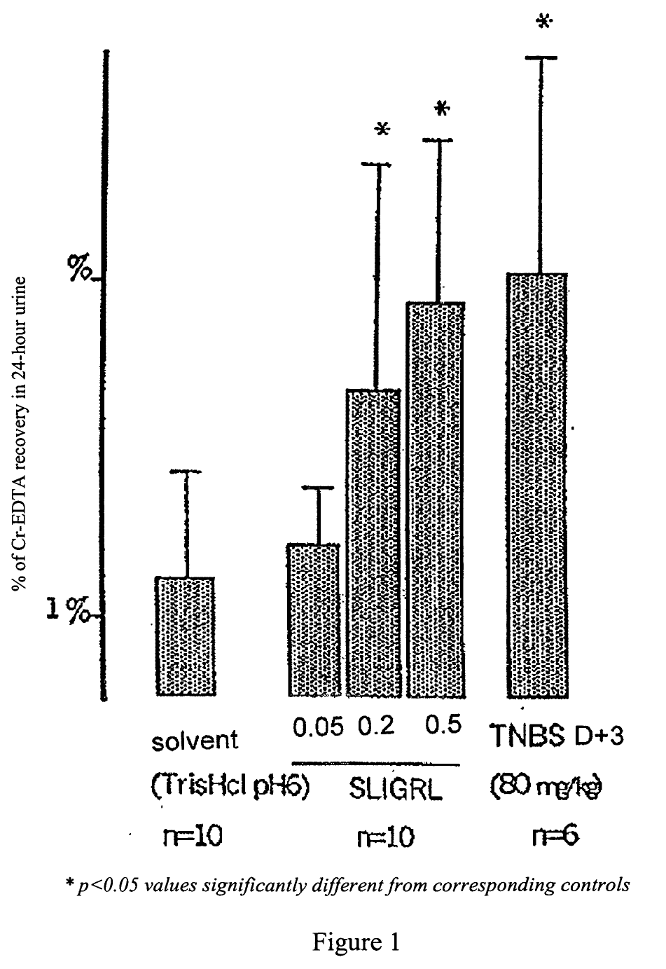

Six groups of eight male Wistar rats (200-250 g) were used for this study. The animals were fitted with an indwelling intracolonic catheter in the proximal colon (3 cm from the caeco-colic junction). In these experiments, four groups of rats received a 10-hour intracolonic infusion of ⁵¹Cr-EDTA (0.5 μ/h). The animals were placed in separate cages and total 24-hour urine was collected. Radioactivity was measured in the urine to evaluate the permeability of the colon mucosa to ⁵¹Cr-EDTA. Increasing doses of the PAR-2 activator peptide (SLIGRL) were injected in the colon lumen (groups 2, 3 and 4) at time t=0 (start of ⁵¹Cr-EDTA infusion) at a dose of 0.05, 0.2 and 0.5 mg/rat. Group 1 received the solvent. FIG. 1 illustrates that SLIGRL induced a dose-dependent increase in permeability to ⁵¹Cr-EDTA, demonstrating that said peptide can increase paracellular permeability.

### Example 2

### Paracellular Permeability in Mouse Colon. Effects of Different Bacterial Protease Inhibitors

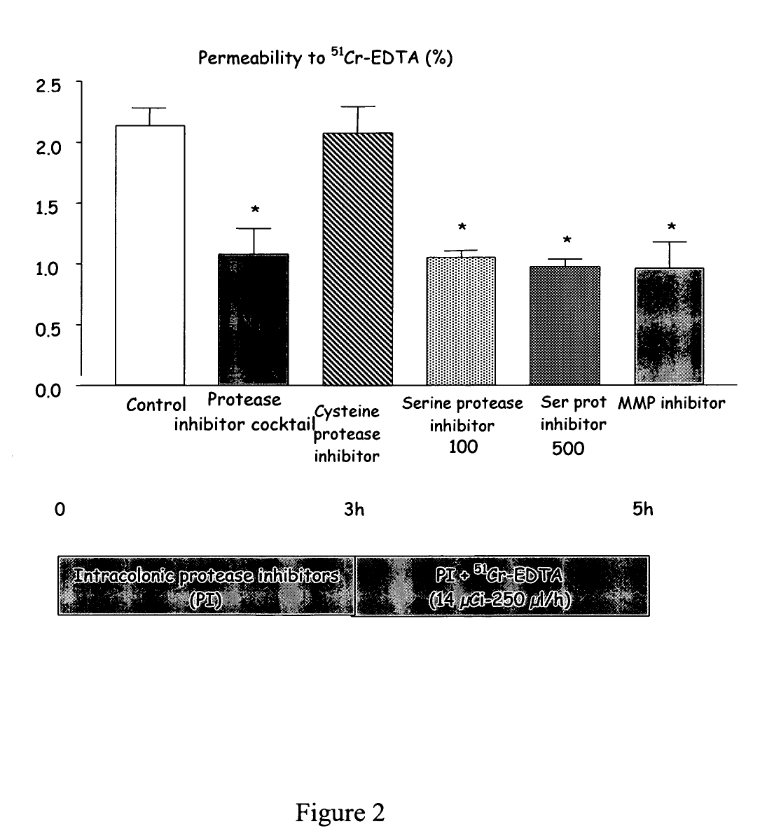

Six groups of ten male Swiss mice (25-30 g) were used for this study. The animals received a 5-hour intracolonic infusion (250 μl/h) of the different protease inhibitor solutions through a catheter inserted through the rectum and fixed at the base of the tail; ⁵¹Cr-EDTA was added to the infusion from hours 3 to 5 (FIG. 2). At t=5 hours, the animals were sacrificed, the colons removed, and total radioactivity remaining in the body was taken as an indicator of paracellular permeability. Paracellular permeability was significantly lower after infusion of the protease inhibitor cocktail (Roche ref.: 1 873 580, EDTA-free), a serine protease inhibitor (aprotinin, Sigma A 1153) and a non-specific matrix metalloprotease inhibitor (galardin, Sigma: M5939.) (FIG. 2)

### Example 3

### Changes in Colon Paracellular Permeability in Mice Induced by Intraluminal Infusion of Trypsin and of Supernatant of Colon Contents and after Treatment with an Antibiotic Cocktail

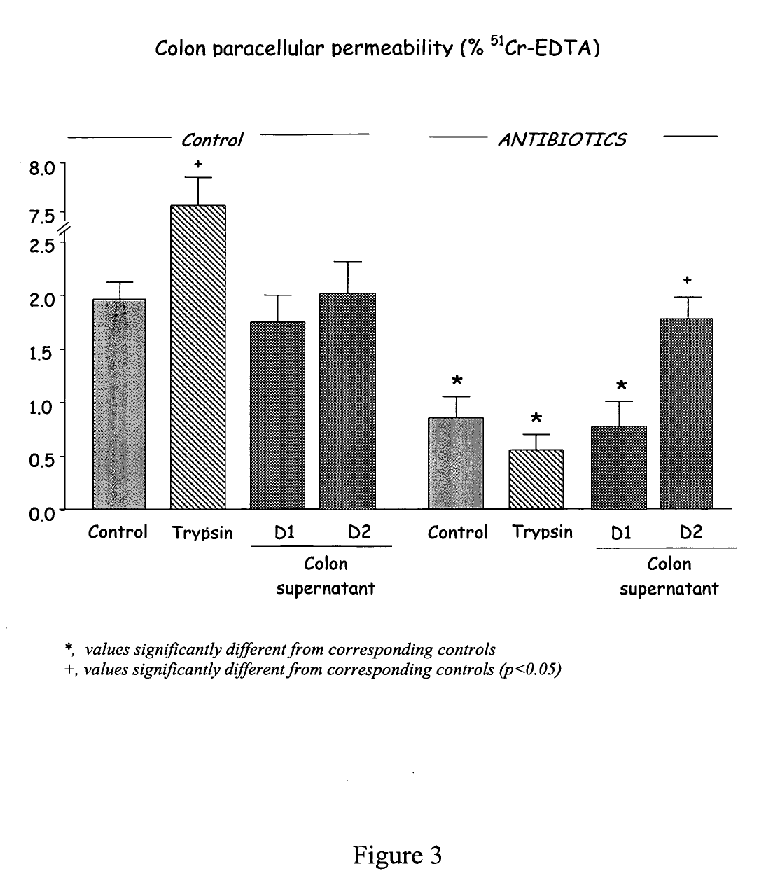

Groups of ten male Swiss mice (Janvier-France) weighing 25-30 g were used for this study. In baseline conditions, the effects on colon paracellular permeability of intracolonic infusion of trypsin (50 μL, 600 U) and of supernatant of colon contents taken from control mice and given by a 3-hour intracolonic infusion (250 μL/h) on one day (D1) or on two consecutive days (D2) were evaluated. The same infusions were then repeated after 12 days of treatment with oral an antibiotic cocktail (neomycin 2 mg/kg/day+ampicillin 1 mg/kg/day). The results show that 1) the antibiotic treatment reduced colon permeability, 2) the increase in permeability induced by trypsin disappeared after the antibiotic treatment which probably induced a loss of epithelial protease receptors, and 3) infusion of colonic supernatant in these animals increased permeability on the second day (FIG. 3).

### Example 4

### Reduction of Rectal Sensitivity to Distention by Intracolonic Infusion of a Protease Inhibitor Cocktail in Rats

In this study, three groups of eight male Wistar rats (250-300 g) were subjected to a rectal distention protocol with incrementally increased (0.4 ml) volumes of 0 to 1.6 ml administered through an embolectomy probe (FORGATY®), after implantation of electrodes in the abdominal striated muscle for electromyographic recording of abdominal contractions known to be a criterion of pain.

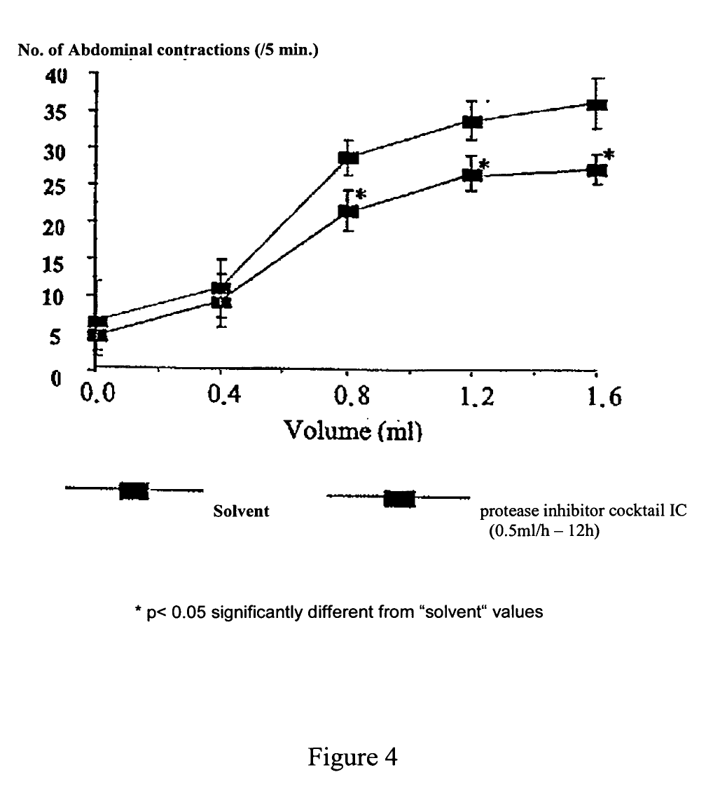

At time t=0 the animals received a 12-hour intracolonic infusion of a protease inhibitor cocktail (Roche ref.: 1 873 580) (2 packets—0.5 ml/h) or its solvent 0.9% NaCl. At the end of the infusion the animals underwent the rectal distention protocol. As compared with the control group, the infusion of protease inhibitors induced a significant decrease in the abdominal response for distention volumes of 0.8, 1.2 and 1.6 ml (FIG. 4), thereby demonstrating that the proteases released by the microflora in the colon lumen play a role in determining the basal state of colorectal sensitivity to distention (FIG. 4).

### Example 5

### Effect of Intracolonic Trypsin Infusion on Rectal Sensitivity to Distention in Rats

In this study, two groups of eight male Wistar rats (250-300 g) were subjected to a rectal distention protocol with incrementally increased (0.4 ml) volumes of 0 to 1.6 ml administered through an embolectomy probe (FORGATY®), after implantation of electrodes in the abdominal striated muscle for electromyographic recording of abdominal contractions known to be a criterion of pain.

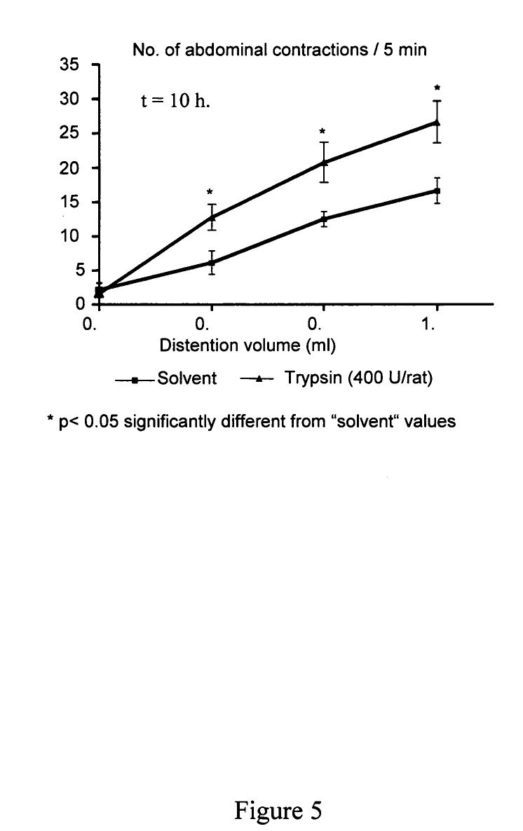

At time t=0, the animals received an intracolonic infusion of trypsin (400 units), a specific PAR-2 receptor activator at the dose of 20 units (group 1) or solvent (group 2). At time t=10 h after the intracolonic trypsin infusion, the animals were subjected to the rectal distention protocol. As compared with the control group, trypsin induced a significant decrease in the abdominal response for distention volumes of 0.8, 1.2 and 1.6 ml (FIG. 5), thereby indicating that an increase in intracolonic protease concentration increases the sensitivity of the colon to distention.

1-17. (canceled)

18. A method for the treatment of functional digestive disorders, comprising administering to a subject in need of such treatment an effective amount of at least one protease inhibitor.

19. The method according to claim 18, wherein the protease inhibitor is an intracolonic protease inhibitor.

20. The method according to claim 18, wherein the protease inhibitor is an inhibitor of serine proteases or an inhibitor of metalloproteases.

21. The method according to claim 18, wherein the protease inhibitor is an inhibitor of serine proteases selected in the group consisting of serpin and derivatives thereof, aprotinin, N-tosyl-L-phenylalanyl chloromethyl ketone, dichloroisocoumarin, nexin-1, AEBSF-HCl, Antipain, benzamidine, leupeptin, TLCK, ovomucoid, phenylmethyl sulfonyl fluoride, PEFABLOC® and soy bean extracts.

22. The method according to claim 18, wherein the protease inhibitor is an metalloprotease inhibitor selected in the group consisting of amastatin, arphamenin, bestatin, diprotin A and phosphoramidon.

23. The method according to claim 18, wherein the protease inhibitor is selected in the group consisting of amprenavir, indinavir, lopinavir, ritonavir, saquinavir, nelfinavir and atazanavir.

24. The method according to claim 18, for controlling paracellular permeability of the intestinal epithelium in subjects with functional intestinal disorders characterized by a state of hyperalgesia.

25. The method according to claim 18, for reducing paracellular permeability of the intestinal epithelium in subjects with functional intestinal disorders characterized by a state of hyperalgesia.

26. The method according to claim 18, for reducing sensitivity to pain in subjects afflicted with or susceptible to functional intestinal disorders characterized by a state of hyperalgesia.

27. The method according to claim 18, for the treatment of functional intestinal disorders (IFD).

28. The method according to claim 18, wherein the functional digestive disorder is an intestinal functional disorder selected in the group consisting of irritable bowel syndrome (IBS), functional abdominal pain without defecation disorder and pain related to food intolerance.

29. The method according to claim 18, wherein the inhibitor is administered by the oral or rectal route.

30. A method for the treatment of hyperalgesia occurring in the context of intestinal pathologies functional digestive disorders, comprising administering to a subject in need of such treatment an effective amount of at least one protease inhibitor.

31. The method according to claim 30, wherein the protease inhibitor is an inhibitor of serine proteases or an inhibitor of metalloproteases.

32. A pharmaceutical composition comprising at least one protease inhibitor and at least one other active agent selected in the group consisting of anticholinergic compounds, prokinetics, antidiarrheals, laxatives, modifiers of motility or visceral sensitivity, in view of a use that is combined, separate or spread out over time.

33. The composition according to claim 32, wherein the protease inhibitor is selected in the group consisting of an inhibitor of serine proteases and an inhibitor of metalloproteases.

34. The composition according to claim 32, wherein said composition is formulated as a capsule or a gelatin capsule which releases its contents by microbial digestion in the colon.
